(19) United States
(12) Patent Application Publication
(10) Pub. No.: US 2011/0245602 A1
(43) Pub. Date: Oct. 6, 2011

(54) FLEXIBLE ENDOSCOPE WITH FULL-LENGTH LUMEN

(76) Inventor: JAMES K. BRANNON, Leawood, KS (US)

(21) Appl. No.: 13/076,408

(22) Filed: Mar. 30, 2011

Related U.S. Application Data
(60) Provisional application No. 61/319,166, filed on Mar. 30, 2010.

(51) Int. Cl. A61B 1/018

(52) U.S. Cl. 600/104

(57) ABSTRACT

The present invention provides a flexible endoscope instrument with a full length lumen extending from a proximal end of a hand section towards an insertion section and out a distal tip end, the insertion section comprising a plurality of lumens covered by a tubular sheath, each of the lumens being axially aligned with each other and at least one lumen being adapted for receiving a surgical instrument therethrough.

### CROSS-REFERENCE TO RELATED APPLICATION

This application claims the benefit of the prior filed U.S. non-provisional application No. 61/319,166 filed Mar. 30, 2010, the contents of which is incorporated herein by reference.

### FIELD OF THE INVENTION

The present invention is broadly directed to improvements in endoscopic surgery and, more particularly, to embodiments of a flexible endoscope instrument with a full length lumen.

### BACKGROUND OF THE INVENTION

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Rigid endoscopic instruments are preferred in situations when precise placement of an instrument is required, as for a surgical procedure. Some endoscopes are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

Conventional endoscopic instruments, particularly those with flexible endoscope sections, provide a surgeon with a large number of functions which can be employed or controlled from a hand section of the instrument. The flexible scope section or insertion section may be provided with a plurality of lumens, some of which are dedicated to specific functions, such as fiberoptic bundles which carry light from a remote source to the surgical site, a coherent fiberoptic bundle which carries an image from the surgical site to a video array within the hand section, lumens which carry fluids to and from the surgical site, and the like. Additionally, one or more tool insertion lumens may be provided for insertion of surgical tools through the instrument to the surgical site. Some flexible endoscope instruments are provided with a steerable tip section which enables the surgeon to selectively curve the tip section by operation of a control on the hand section to selectively observe or surgically access a particular location at the surgical site. Details of representative configurations of flexible endoscope instruments of the type described above can be found in U.S. Pat. Nos. 4,909,142; 6,569,087; and 6,773,395 which are incorporated herein by reference.

Each of the functions available to the surgeon requires a fluid or electrical connection to an external device and/or a control of some sort. As a result, the hand section of a typical flexible endoscope instrument can be crowded with connections and controls. In order to provide access to a lumen for surgical instruments, flexible endoscopes typically provide a tool access port at a distal location on the hand section which is oriented at an angle to an axis of the flexible scope section. The angled tool access port includes an angled lumen segment which is joined with a main tool lumen at an angle or which curves into communication with the main tool lumen which extends axially along the flexible scope section. The main tool lumen may also function as a fluid management lumen which extends into the hand section to connect with sources of irrigation fluids and/or suction. In such a case, the angled or branch lumen section joins the main lumen at an angle. The angled tool port may include means for capping the port, such as threads or the like, to control the outflow of fluids from a surgical site.

In the use of endoscopic instruments, it is often necessary for the surgeon to locate the surgical site indirectly, that is, without a direct view of the site initially. This is especially true with regard to endoscopic and arthroscopic surgery. It has often been necessary to employ radiopaque endoscopic instruments and a radiant imaging technique, such as fluoroscopy or computed tomography to extend an endoscopic instrument from an external incision to the surgical site. Direct viewing of an anatomical site is often necessary for more conclusive diagnosis of the condition of tissue at the site and for surgical intervention or treatment at the site. In the case of arthroscopic or endoscopic surgery, direct viewing is sometimes accomplished using a second endoscopic instrument which enters at a different angle from a scope through which a surgical instrument will be passed. This is referred to as triangulation.

Because endoscopic surgery is intended to be minimally invasive, it is desirable to limit the number of incisions made in the patient and to limit irradiation of the patient to correctly position instruments at the surgical site. Therefore, once an incision is made and a path from the incision to the surgical site has been established, every effort is made to limit additional incisions and imaging irradiations to position the ends of instruments at the surgical site. Often, the first instrument extended from an incision to a surgical site is a guide wire, the tip of which is often placed with aid of a radiant imaging technique, such as fluoroscopy or computed tomography. Afterwards, an endoscopic instrument, such as a trephine, trocar, portal instrument, or the like is telescoped over the guide wire to guide a distal tip of the instrument to the surgical site. Once the instrument is in place, the guide wire can be removed to enable the insertion of additional instruments or the exchange of fluids through the instrument. If a different endoscopic instruments needs to be inserted to the site, the guide wire can be reinserted through the instrument in place and the instrument removed to enable a subsequent instrument to be guided to the surgical site. Thus, each instrument provides a guide from the incision to the surgical site to the next instrument which needs to be inserted or to a guide wire for such a next instrument.

A problem occurs with the use of a flexible endoscope with a steerable tip on which the only available lumen terminates proximally at an angled port. Conventional flexible endoscope instruments typically have such complex hand sections that a lumen extending entirely through the flexible section and the hand section is not provided. The types of guide wires that are employed in endoscopic surgery are typically relatively stiff, depending on their gauge or thickness, and formed of nitinol, a nickel titanium alloy. Although it might be possible to insert some guide wires through an angled port and extend it through the instrument to the distal tip, it is not possible to reverse the procedure, that is, introduce a guide wire through the distal tip and find the branch lumen to exit the angled port. Thus, it is not possible to effectively use a guide wire to guide the distal tip of a conventional flexible endoscope instrument through an incision to an endoscopic surgical site and remove the guide wire from the proximal end to make alternative use of the lumen through which the guide wire extends. Other means must be employed to guide the distal tip of a flexible endoscope to a surgical site. Such means may include a portal scope large enough for the insertion section of the flexible endoscope to extend through. However, a larger portal scope might require a larger incision and result in greater injury to the patient's tissues during surgery. Alternatively, radiant imaging could be used, but this increases the patient's exposure to the radiant energy.

### SUMMARY OF THE INVENTION

The present invention provides improvements in endoscopic instruments by providing a flexible endoscope instrument with a full length lumen extending axially from a distal tip of an insertion section of the instrument to a proximal end of a hand section of the instrument.

An embodiment of the invention provides an endoscope instrument including a hand section which is grasped by the surgeon to manipulate the instrument and a flexible scope section terminating in a distal tip. The instrument is provided with lumens through which extend fiberoptic bundles to carry light from a remote light source to the surgical site and a coherent fiberoptic bundle carrying an image from the surgical site to a video image array mounted within the hand section. The instrument may also be provided with a steerable tip section near the distal end tip and including a steering mechanism controlled by manipulation of a tip steering control provided on the hand section. The instrument may also include one or more open lumens through which an irrigant may be pumped to the surgical site or which may be connected to a suction source to draw fluids from the surgical site. The hand section of the instrument may be provided with valves to control the flow of fluids to or from the surgical site with controls to operate such valves. Alternatively, the open lumen may be used for inserting a flexible surgical tool for remotely performing a surgical operation at the surgical site. In particular, at least one of the open lumens extends entirely through the endoscope instrument and opens at a proximal or upper end of the hand section of the instrument. Additionally, all portions of the full length lumen are in alignment to enable a guide wire to extend entirely through the instrument. The open lumen may be provided with an angled branch in the hand section terminating in an angled port. The angled branch provides an alternative entry point for a flexible surgical instrument to be extended through the endoscope instrument.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a fragmentary elevational view of an embodiment of a flexible endoscope instrument with a full length lumen according to the present invention.

FIG. 2 is an elevational view of the flexible endoscope instrument at a reduced scale and illustrates a guide wire extending entirely through the instrument and exiting both a proximal end and a distal end of the instrument.

FIG. 3 is an enlarged fragmentary perspective view of a distal tip of the endoscope instrument with portions broken away to illustrate various exemplary functional features of the instrument.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a flexible endoscope instrument with a full length lumen according to the present invention. The instrument 1 generally includes a rigid hand section 2 from which a flexible scope or insertion section 3 extends.

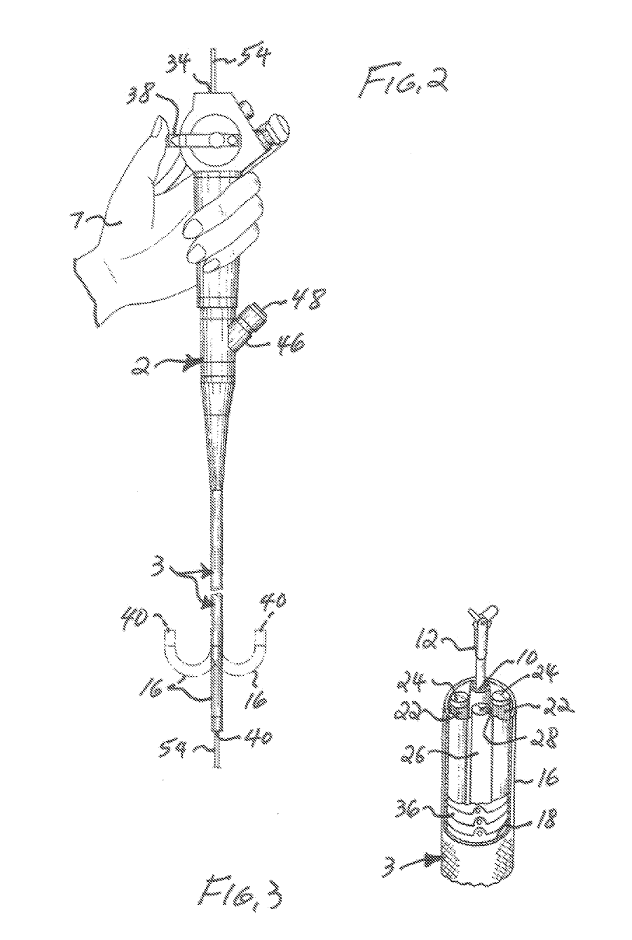

The illustrated endoscope hand section 2 is a housing or enclosure which is sized for a comfortable grip by the hand 7 of a surgeon or other surgical personnel for supporting and manipulating the instrument 1. The flexible insertion section 3 extends from a lower or distal end of the hand section 2. The flexible insertion section 3 is a bundle of a plurality of lumens, some dedicated to selected functions, and at least one lumen 10 provided for the insertion of surgical tools 12 (FIG. 3) through the instrument 1 to a surgical site. In the instrument 1 of the present invention, the lumen 10 is a full length lumen which extends entirely through the instrument 1 from a proximal or top end 14 of the hand section 2 and through the flexible section 3 to a distal tip section 16 (FIGS. 2 and 3) thereof. All parts of the lumen 10 are axially aligned with one another, as will be described further below. The bundle of lumens within the flexible insertion section 3 is covered by a flexible tubular sheath 18 (FIG. 3).

The illustrated insertion section 3 includes one or a pair of elongated fiberoptic bundles 22 terminating in dispersion lenses 24 for carrying and dispersing light at a surgical site to illuminate the site. The insertion section 3 also includes an elongated coherent fiberoptic bundle 26 and an image gathering lens 28 for capturing an image or moving images of the surgical site and carrying such images to an image array (not shown) positioned within the hand section 2. An image connector (not shown) is provided for accessing circuitry associated with the image array for displaying such images on a video display unit or monitor (not shown) in a conventional manner. The insertion section 3 may also include one or more lumens, such as the lumen 10, for the insertion of surgical tools or instruments 12 therethrough to the surgical site, such as the remotely controlled forceps 12 illustrated in FIG. 3. It is foreseen that the insertion section 3 may include additional lumens (not shown) for surgical site fluid management, such as for pumping irrigants or other fluids to the surgical site, by way of fluid conduits 30 connected to the hand section 2, and extracting such fluids therefrom by external suction, under the control of valves (not shown) positioned within the hand section 2 and fluid control buttons 32 positioned on the hand section 2. Alternatively, surgical site fluid management may be implemented through the full length lumen 10 by way of passages (not shown) communicating therewith. In some cases, it may be necessary to plug the proximal end or end port 34 of the lumen 10 to avoid suction leakage or the outflow of fluids from the lumen 10.

The illustrated endoscope instrument 1 is a flexible endoscope instrument and may have a steerable tip section 16 which can be selectively curved to enable guiding the tip section 16 and the flexible insertion section 3 from an external incision to a surgical site or to afford a surgeon a better view of tissues, the condition of such tissues, and the general anatomy at a surgical site. Thus, the tip section 16 illustrated instrument 1 includes a tip steering mechanism 36, of a conventional configuration, which is operated using a tip steering control 38 mounted on the hand section 2 by way of one or more control cables (not shown) extending through the insertion section 3 to selectively position the distal tip 40 of the tip section 16.

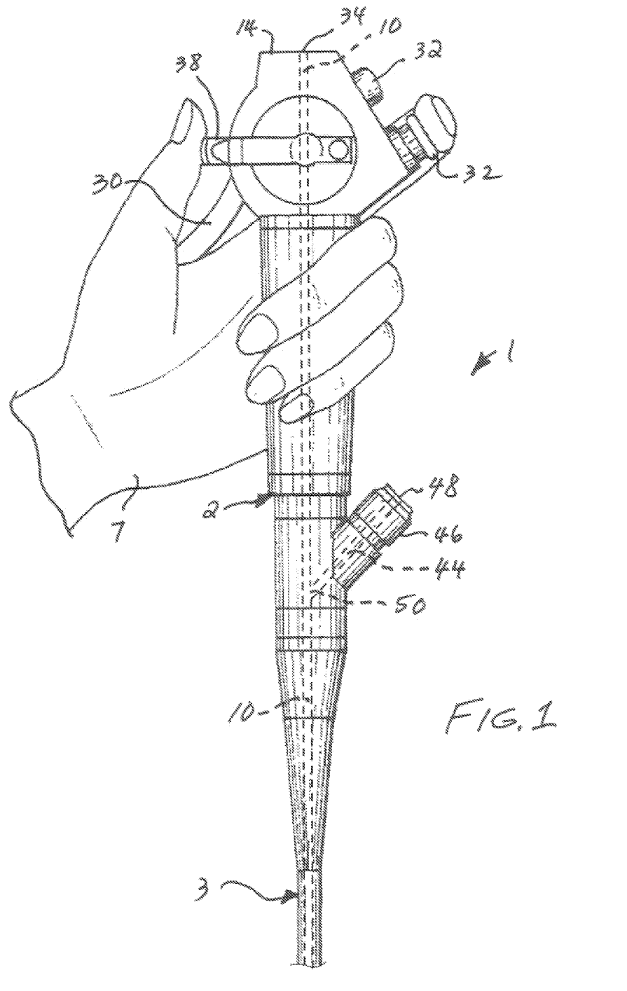

The illustrated full length lumen 10 includes an angled branch lumen 44 extending through a branch fitting 46 extending from the hand section 3 and terminating in an angled branch port 48. The branch lumen 44 communicates with the lumen 10 at an angled junction 50 (FIG. 1). The angled branch port 48 provides for the insertion of remotely operated, flexible surgical instruments 12, such as flexible forceps and the like. Under some circumstances, it may be necessary to plug the branch port 48 for fluid management purposes, as described above with respect to the end port 34.

The illustrated flexible endoscope instrument 1 can be used in endoscopic, arthroscopic, or laparoscopic surgery in the same manner as conventional flexible endoscope instrument. However, the endoscopic instrument 1 has the added advantage of effectively being guided through an external incision to a surgical site using a previously placed guide wire 54. With a conventional endoscope, provided with a branch lumen similar to the branch lumen 44, it is not possible to locate the angled junction 50 to extend the guide wire 54 out the branch port 44. However, the illustrated full length lumen 10 is formed by a proximal or upper lumen section 58, extending from the lumen junction 50 to the end port 34, and a distal or lower lumen section 60, extending from the lumen junction 50 to the distal tip 40. The proximal and distal lumen sections 58 and 60 are aligned at the lumen junction 50 such that a guide wire 54 entering the distal end of the lumen 10 can pass entirely through the instrument 1 and out the end port 34. This enables the guide wire 54 to act as a guide for the tip section 16 to the surgical site and removal of the guide wire 54 from the instrument 1 to enable further use of the lumen 10, as for fluid management or insertion of a remotely controlled surgical instrument 12.

If it should be necessary to replace the flexible endoscope instrument 1 at the surgical site with another endoscopic instrument, the guide wire 54 can be inserted through the end port 34 and passed through the instrument 1 and out the distal end of the lumen 10 at the surgical site. The insertion section 2 can then be removed from the patient and replaced by another endoscopic instrument, using the thus emplaced guide wire 34.

It is to be understood that while certain forms of the present invention have been illustrated and described herein, it is not to be limited to the specific forms or arrangement of parts described and shown.

What is claimed and desired to be secured by Letters Patent is:

1. A flexible endoscope instrument with a full length lumen substantially extending therethrough, the lumen extending from a proximal end of a hand section towards an insertion section and out a distal tip end, said insertion section comprising a plurality of lumens covered by a tubular sheath, each of said lumens being axially aligned with each other and at least one lumen being adapted for receiving a surgical instrument therethrough.

2. The flexible endoscope instrument of claim 1 further comprising:
an upper lumen section extending from an angled lumen junction to an end port;
a lower lumen section extending from said lumen junction to said distal tip;
said upper and lower lumen sections being aligned at said lumen junction for passing a second surgical instrument received at said distal tip through said instrument and out said instrument at an end port, and
said second surgical instrument guiding said distal tip towards a surgical site, whereby said second surgical instrument is removable from said instrument at said end port.

3. The flexible endoscope instrument of claim 2 wherein said second surgical instrument is insertable at said end port through said upper and lower lumen sections out said instrument at said end port for removal of said instrument from said surgical site.

4. The flexible endoscope instrument of claim 1 further comprising a tip steering control mounted on the hand section by at least one control cable extending through said insertion section to selectively position said distal tip.

5. The flexible endoscope instrument of claim 1 further comprising a replaceable end port being adapted for management of fluid at a surgical site.

6. The flexible endoscope instrument of claim 1 further comprising an angled branch lumen terminating at an angled branch port, said angled branch lumen extending between from said hand section towards said branch fitting, and said angled branch port receiving a remotely operated surgical instrument.

7. The flexible endoscope instrument of claim 6 wherein said remotely operated surgical instrument is a flexible forcep.

8.
